(19) United States
(12) Patent Application Publication
(10) Pub. No.: US 2003/0195489 A1
(43) Pub. Date: Oct. 16, 2003

(54) Coupling syringe system and methods for obtaining a mixed composition

(75) Inventor: Kenneth S. Peterson, Lancaster, PA (US)

Correspondence Address: SCHWEGMAN, LUNDBERG,, WOESSNER & KLUTH, P.A., P.O. Box 2938, Minneapolis, MN, 55402, US

(73) Assignee: Atrix Laboratories, Inc.

(21) Appl. No.: 10/456,814

(22) Filed: Jun. 6, 2003

Related U.S. Application Data
(62) Division of application No. 09/405,463, filed on Sep. 24, 1999.

(51) Int. Cl. A61M 31/00

(52) U.S. Cl. 604/500; 604/82; 366/268; 366/130

(57) ABSTRACT

The present invention relates generally to medical devices for mixing, preparing and administering therapeutic compositions, and more particularly to a system comprising two syringes and a locking ring wherein two compositions are mixed between the two syringes immediately prior to administration.

### RELATED APPLICATIONS

This application is a divisional of U.S. patent application Ser. No. 09/405,463, filed Sep. 24, 1999, which is incorporated herein by references.

### BACKGROUND OF THE INVENTION

A large number of drugs and other medicaments are routinely prepared and administered to patients in a health care facility. Sometimes, two components of a therapeutic composition are required to be mixed immediately prior to administration. One method for mixing therapeutic compositions immediately prior to administration includes mixing two solutions in a mixing vessel, whereupon the mixture is drawn into a syringe, and the resulting mixed composition is then applied to an appropriate site of the patient. However, this process is often cumbersome, a significant amount of the drug may be lost, and the time between the mixing and the application is often too long with sensitive compositions (i.e., compositions that, upon mixing, must be immediately administered).

Another method for mixing therapeutic compositions immediately prior to administration employs two syringes that are clamped together. Output ends of the syringes are inserted into a Y-shaped coupling device having two input openings and a single output opening. Mixing occurs in the Y-shaped coupling device or in a needle connected to the single output end of the Y-shaped coupling device. However, with this type of arrangement, control over the degree of mixing does not exist and the degree of mixing, therefore, may be inadequate.

Another method for mixing therapeutic compositions immediately prior to administration includes coupling two syringes with an independent coupling means, thereby allowing the contents of one syringe to be mixed with the contents of the other coupled syringe. The independent coupling means, however, provides a space where there is very little agitation due to plug flow of the contents. The contents, therefore, do not mix well. Additionally, when the syringes are uncoupled (i.e., disengaged), the contents have to be aspirated out of the independent coupling means or they will be lost. In addition, the independent coupling means must be removed and discarded before attaching a needle to the delivery or injection syringe.

Thus, there is a need for a syringe system wherein components of a composition can be easily mixed by the end user without losing a significant amount of mixed composition during the mixing process and wherein the mixed composition can be easily and rapidly administered to a patient.

### SUMMARY OF THE INVENTION

The present invention provides a coupling syringe system for obtaining a mixed composition. Included are a first syringe having a male end portion and a second syringe having a female end portion. The male end portion of the first syringe includes a locking ring which is configured to detachably connect to the female end portion of the second syringe. The male end portion of the first syringe can be detachably connected to the female end portion of the second syringe via a locking ring.

The present invention also provides a syringe having a female end portion which is configured to detachably connect to a male end portion of a second syringe via a locking ring of the second syringe.

The present invention also provides a method for forming a mixed composition. The method includes providing a first composition within a first syringe having a male end portion wherein the male end portion includes a locking ring which is configured to detachably connect to a female end portion of a second syringe. A second composition is provided within the second syringe and the female end portion of the second syringe is configured to detachably connect to the male end portion of the first syringe via the locking ring. The female end portion of the second syringe is connected to the male end portion of the first syringe via the locking ring such that the first composition and the second composition are in communication. Then, at least a portion of the first composition is forced into the second syringe or at least a portion of the second composition is forced into the first syringe, so as to provide a mixed composition.

The present invention also provides a method for administering a mixed composition to a patient. This method includes providing a first composition within a first syringe having a male end portion wherein the male end portion includes a locking ring which is configured to detachably connect to a female end portion of a second syringe. A second composition is provided within the second syringe having the female end portion which is configured to detachably connect to the male end portion of the first syringe via the locking ring. The female end portion of the second syringe is connected to the male end portion of the first syringe via the locking ring such that the first composition and the second composition are in communication. Then, at least a portion of the first composition is forced into the second syringe or at least a portion of the second composition is forced into the first syringe, so as to provide a mixed composition. The first and second syringes are disconnected and the male end portion of the first syringe is connected, via the locking ring, to a discharge assembly configured to detachably connect to the locking ring. The discharge assembly is inserted into the patient. At least a portion of the mixed composition is forced through the discharge assembly, so as to administer the portion of the mixed composition to a patient.

### BRIEF DESCRIPTION OF THE DRAWINGS

FIG. 1 illustrates a coupled syringe system with a mixed composition formed from introducing the composition of the syringe with the male end portion into the syringe with the female end portion.

FIG. 2 illustrates a coupled syringe system with a mixed composition formed from introducing the composition of the syringe with the female end portion into the syringe with the male end portion.

FIG. 3 illustrates a syringe with a female end portion.

FIG. 4 illustrates a syringe with a male end portion.

FIG. 5 illustrates a coupled syringe system with a mixed composition formed from introducing the composition of the syringe with the female end portion into the syringe with the male end portion.

FIG. 6 illustrates a syringe with a male end portion detachably connected to a discharge assembly wherein the discharge assembly includes a needle and wherein a needle cover is removably mounted over the needle cannula.

### DETAILED DESCRIPTION OF THE INVENTION

The coupling syringe system of the present invention allows for the effective mixing of compositions immediately prior to administration. The mixing does not result in a significant loss of the composition. In addition, the time between the mixing and the administration of the composition is minimal, such that a sensitive composition (i.e., a composition that, upon mixing, must be immediately administered) is not chemically or physically altered (i.e., there is minimal decomposition). The use of the coupling syringe system of the invention does not result in a plug flow of the contents. In addition, the coupling syringe system can conveniently be disassembled and a needle can conveniently be attached to the syringe which includes a male end portion and a locking ring.

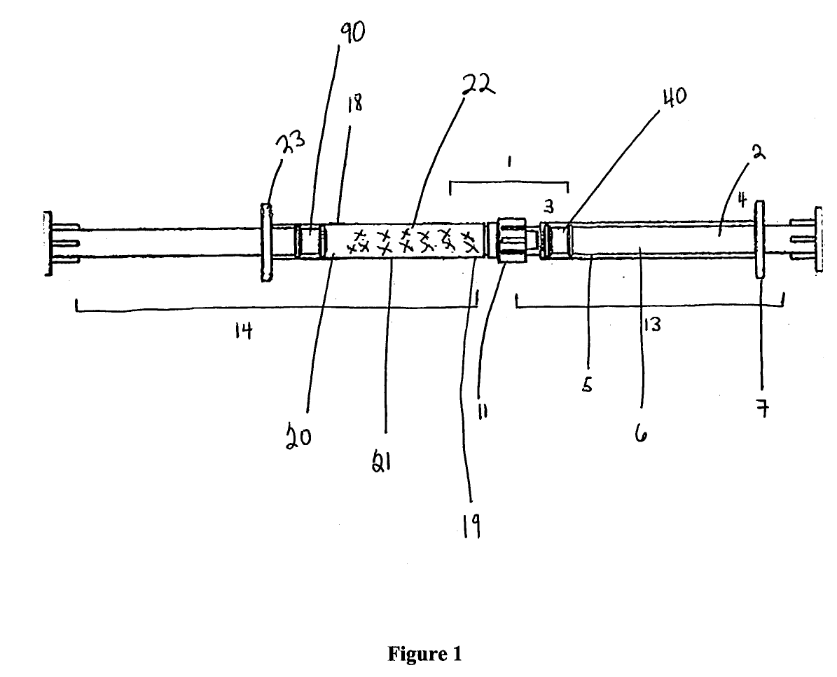

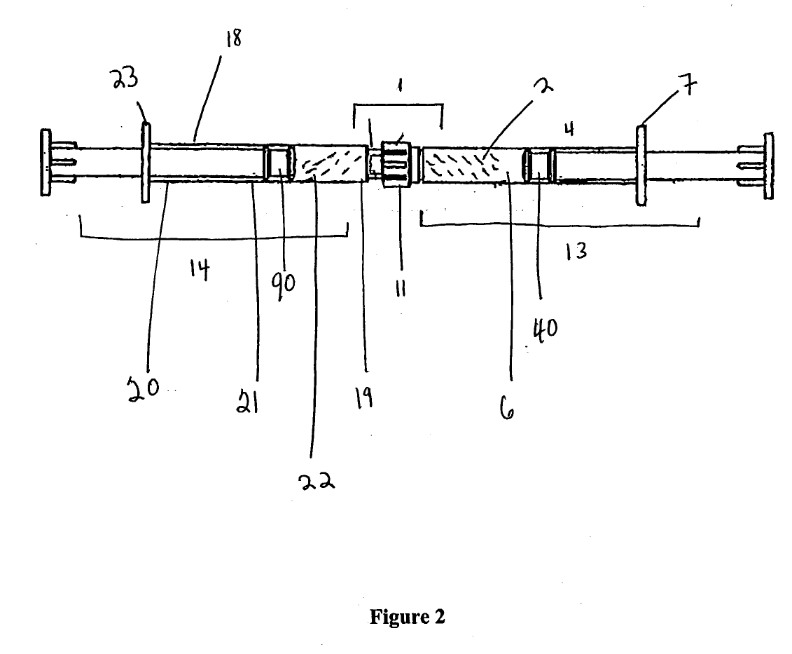

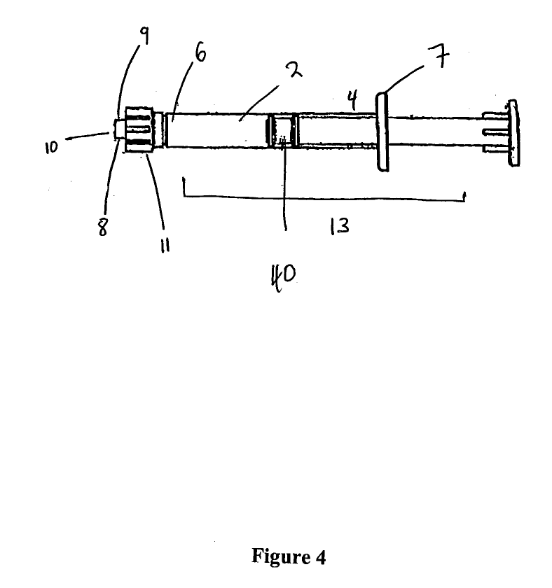

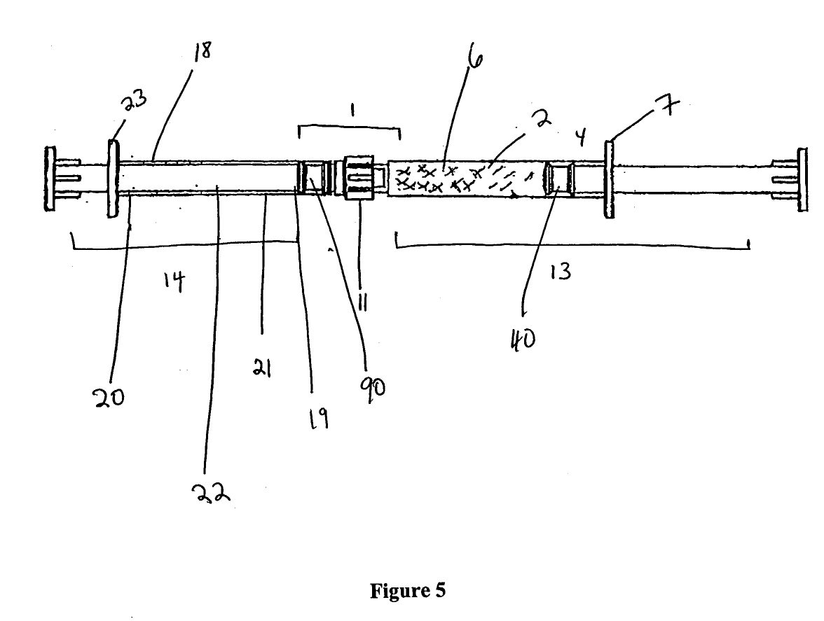

Referring to FIGS. 1-6, a coupling syringe system of the present invention is identified generally by the numeral 1. As shown in FIGS. 1, 2, and 5, syringe system 1 includes a first syringe 13 and a second syringe 14. The first syringe 13 includes a barrel 2. The barrel 2 has a distal end 3, an open proximal end 4, and a generally cylindrical wall 5 extending between the ends to define a fluid receiving chamber 6. Cylindrical wall 5 of the first syringe barrel defines an outside diameter along much of its length. An outwardly projecting finger flange 7 is defined near proximal end 4 of the first syringe barrel 2 for facilitating digital manipulation of the first syringe. Additionally, as shown in FIG. 4, distal end 3 of the first syringe barrel 2 is characterized with a tip 8. Tip 8 is provided with a fluid passage 9 extending therethrough and communicating with fluid receiving chamber 6. The tip 8 is also provided with a male end portion 10 wherein the male end portion 10 is provided with a locking ring 11.

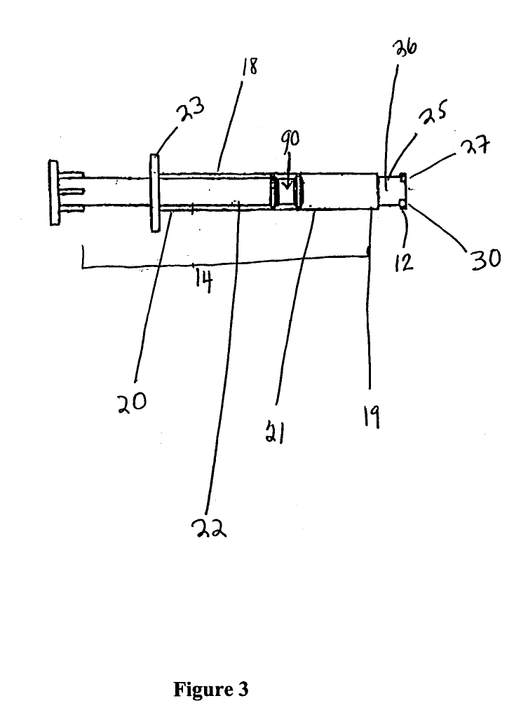

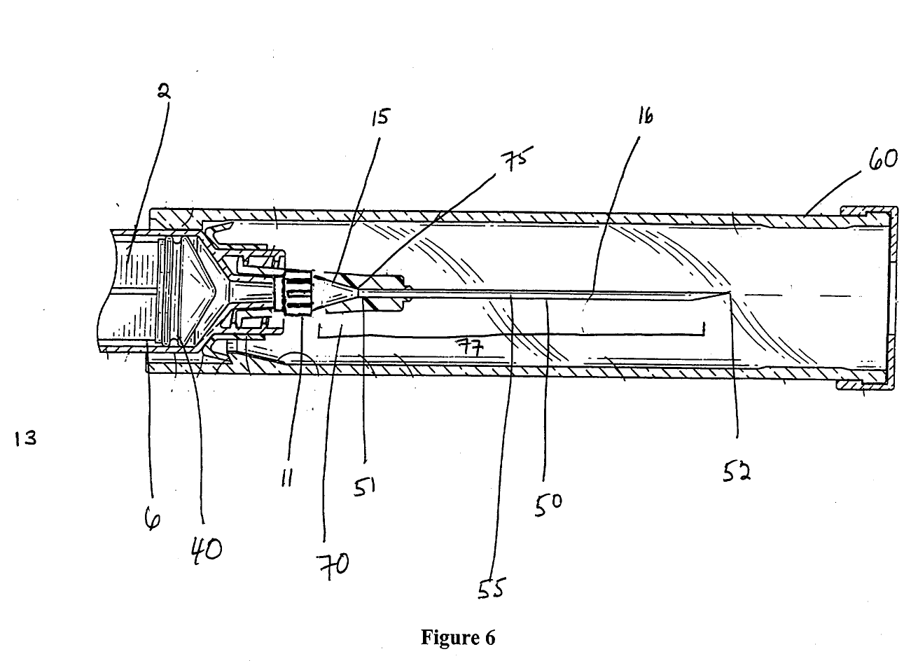

The locking ring 11 is configured such that the interior of the locking ring 11 contains threads which are adapted to receive protruding members 12 exteriorly disposed on the female end portion of the second syringe 14 as shown in FIG. 3. The locking ring 11 is designed to interlock the first syringe 13 (i.e., the syringe including the male end portion) and the second syringe 14 (i.e., the syringe including the female end portion). In addition, the locking ring 11 is configured to detachably connect to a discharge assembly 15. Specifically, the discharge assembly 15 can include a needle 16 (see FIG. 6).

As shown in FIGS. 1, 2, and 5, syringe system 1 also includes a second syringe 14 having a barrel 18. The barrel 18 has a distal end 19, an open proximal end 20, and a generally cylindrical wall 21 extending between the ends to define a fluid receiving chamber 22. Cylindrical wall 21 of the second syringe barrel defines an outside diameter along much of its length. An outwardly projecting finger flange 23 is defined near proximal end 20 of the second syringe barrel 18 for facilitating digital manipulation of the second syringe 14. As shown in FIG. 3, distal end 19 of the second syringe barrel is characterized with a tip 25. Tip 25 is provided with a fluid passage 26 extending therethrough and communicating with fluid receiving chamber 22. The tip 25 is also provided with a female end portion 27 wherein the female end portion 27 is configured to detachably connect to the locking ring 11. The female end portion 27 includes one or more (e.g., 1, 2, 3, or 4) exteriorly protruding members 30 adapted to detachably engage the locking ring 11. The protruding members 30 are configured such that they can thread into the locking ring 11.

As shown in FIGS. 1, 2, 4, and 6, a plunger 40 is disposed in fluid receiving chamber 6 and is in sliding fluid-tight engagement with cylindrical wall 5 of syringe barrel 2. Sliding movement of plunger 40 in a distal direction causes the composition (i.e., solid, liquid, or mixture thereof) in chamber 6 to be expelled through passage 9 of tip 8 (see FIG. 4) and into fluid receiving chamber 22 (see FIGS. 1, 2, 3, and 6) thereby mixing the composition (i.e., solid, liquid, or mixture thereof) of chamber 6 with the composition (i.e., solid, liquid, or mixture thereof) of chamber 22. Conversely, sliding movement of plunger 40 in a proximal direction draws the composition (i.e., solid, liquid, or mixture thereof) in chamber 22 through passage 26 and into fluid receiving chamber 6 thereby mixing the composition (i.e., solid, liquid, or mixture thereof) of chamber 26 with the composition (i.e., solid, liquid, or mixture thereof) of chamber 22. It is appreciated that those skilled in the art understand that any combination of the above steps can be carried out and repeated until such time as an effective amount of mixing is attained.

As shown in FIGS. 1, 2, 3, and 6, a plunger 90 is disposed in fluid receiving chamber 22 and is in sliding fluid-tight engagement with cylindrical wall 21 of syringe barrel 18. Sliding movement of plunger 19 in a distal direction causes the composition (i.e., solid, liquid, or mixture thereof) in chamber 22 to be expelled through passage 26 of tip 25 and into fluid receiving chamber 6 (see FIGS. 1, 2, 4, and 6) thereby mixing the composition (i.e., solid, liquid, or mixture thereof) of chamber 22 with the composition (i.e., solid, liquid, or mixture thereof) of chamber 6. Conversely, sliding movement of plunger 90 in a proximal direction draws the composition (i.e., solid, liquid, or mixture thereof) in chamber 6 through passage 9 and into fluid receiving chamber 22 thereby mixing the composition (i.e., solid, liquid, or mixture thereof) of chamber 6 with the composition (i.e., solid, liquid, or mixture thereof) of chamber 22. It is appreciated that those skilled in the art understand that any combination of the above steps can be carried out and repeated until such time as an effective amount of mixing is attained.

As shown in FIG. 6, a discharge assembly 15 can be connected to the locking ring 11 of the first syringe 13. More particularly, the discharge assembly 15 includes needle cannula 50 having a proximal end 51, a sharp distal end 52 and a lumen 55 extending therebetween. A hub 75 joined to the cannula so that the lumen 55 is in fluid communication with the hub 75. Tip 8 fits into hub 75 and frictionally engages the hub 75 so that the lumen 55 of needle cannula communicates with passage through tip 8 and further communicates with fluid receiving chamber 6 of syringe barrel 2. In this embodiment, needle assembly 77 is removably mounted to tip 8. However, it is within the purview of the present invention to include a needle cannula that is directly and permanently mounted to the syringe tip.

As shown in FIG. 6, a needle cover 60 can be removably mounted over needle cannula 50 to prevent accidental sticks prior to use of syringe assembly 70. Needle cover 60 can be removed from syringe assembly 70 immediately prior to use.

In an alternative embodiment, a first securing device can conveniently be mounted on the interior surface of barrel 2 or on the inside of barrel 18. The first securing device, upon engaging with a second securing device mounted on the external surface of plunger 40 or plunger 90, respectively, can prohibit the plunger 40 or the plunger 90 from disengaging from barrel 2 or the barrel 18, respectively.

The first syringe and the second syringe can conveniently be manufactured from any suitable material. Typically, both the first syringe and the second syringe are each independently manufactured from glass or plastic (e.g., polypropylene, polyethylene, polycarbonate, polystyrene, and the like).

The size of both the first syringe and second syringe can independently be any suitable size. Suitable sizes include a syringe barrel of about 0.01 to about 100 cc, about 0.1 cc to about 50 cc, about 0.1 cc to about 25 cc, or about 0.5 cc to about 10 cc.

The first syringe 13 (i.e., the syringe including the male end portion and locking ring) can conveniently be manufactured by any suitable process. The first syringe can conveniently be manufactured by an injecting molding process where the entire syringe is made as one unit. Alternatively, the first syringe can be manufactured by independently molding the syringe and locking ring and then mounting (i.e., attaching) the locking ring and first syringe. Preferably, the locking ring is permanently attached to the first syringe. Although the ring can also be mounted coaxially and rotably with tip 8 by a flange and seal configuration. In this configuration, the ring can be rotated around the tip. Typically, the locking ring is permanently attached to the first syringe by welding the two pieces together.

The second syringe 14 (i.e., the syringe including the female end portion) can conveniently be manufactured by any suitable process. The second syringe can be manufactured by an injecting molding process where the entire syringe is made as one unit.

Each composition to be combined with a syringe can independently be a solid, liquid, or mixture thereof. In addition, the solid can be a powder or crystalline material. As used herein, a mixture of a solid and a liquid can be a heterogeneous phase (e.g., an emulsion or a colloidal suspension). Alternatively, a mixture of a solid and a liquid can be a homogeneous phase (i.e., a solid completely dissolved in a liquid).

Each composition can independently includes one or more (e.g., 1, 2, or 3) compounds. In addition, the compound of the composition can be a drug delivery system, a drug (i.e., pharmaceutical) or a pharmaceutically acceptable salt thereof, a liquid carrier, a liquid, a lipid formulation, or a vaccine.

Any suitable drug delivery system can be employed. A suitable drug delivery system includes, but is not limited to, is the Atrigel Ò delivery system mixed with doxycycline or leuprolide acetate. The Atrigel Ò system is described in U.S. Pat. No. 5,278,201, the disclosure of which is incorporated herein by reference.

Any suitable drug (i.e., pharmaceutical) or pharmaceutically acceptable salt thereof can be employed. Suitable classes of drugs include antibiotics, peptides, hormones, analgesics, growth factors, and any agent described in US. Pat. No. B14938763, the disclosure of which is incorporated herein by reference. The drug can exist as a solid (e.g., crystal or powder), an oil, or as a liquid. In addition, the drug may exist in a microcapsule containing the drug or as a microparticle.

Any suitable liquid carrier can be employed. Suitable liquid carriers include a collagen solution, a sterile aqueous solution, a sterile saline solution, an alcoholic solution, or any suitable mixture thereof. In addition, the liquid carrier can be an emulsion formed from a mixture of a sterile aqueous solution and a sterile saline solution.

Specifically, the liquid drug delivery system can be the Atrigel Ò system mixed with a powder drug (e.g., doxycycline or leuprolide acetate).

Specifically, the drug can be an antibiotic or growth factor.

Specifically, the liquid carrier can be a collagen solution and a powder drug.

Specifically, the liquid carrier can be a sterile aqueous or a sterile saline solution and a powder drug.

Specifically, the liquid can be an alcohol and a drug mixed with a sterile saline or a sterile aqueous solution.

Specifically, the lipid formulation can be mixed with a sterile aqueous solution or a sterile saline solution to form an emulsion.

Specifically, the liquid carrier (e.g., a sterile aqueous solution or a sterile saline solution) can be mixed with a microcapsule or a microparticle containing drug.

Specifically, the vaccine solution can be mixed with an oil to form an emulsion.

Any suitable method of administration can be employed. Typically, the mixed composition can be administered to a patient by intravenous, intramuscular, intraperitoneal, or subcutaneous routes.

What is claimed:

1. A coupling syringe system for obtaining a mixed composition comprising a first syringe having a male end portion and a second syringe having a female end portion, wherein the male end portion comprises a locking ring configured to detachably connect to the female end portion of the second syringe.

2. The coupling syringe system of claim 1 wherein the female end portion comprises one or more exteriorly protruding members adapted to detachably engage the locking ring.

3. The coupling syringe system of claim 1 wherein the locking ring is configured to detachably connect to a discharge assembly.

4. The coupling syringe system of claim 3 wherein the discharge assembly comprises a needle.

5. The coupling syringe system of claim 1 wherein the female end portion of the second syringe is detachably connected to the male end portion of the first syringe via the locking ring.

6. The coupling syringe system of claim 1 wherein the female end portion of the second syringe is detached from the male end portion of the first syringe.

7. A syringe having a female end portion wherein the female end portion is configured to detachably connect to a male end portion of a second syringe via a locking ring of the second syringe.

8. The syringe of claim 7 wherein the female end portion comprises one or more exteriorly protruding members adapted to detachably engage the locking ring.

9. A method for forming a mixed composition wherein a first syringe containing a first composition is directly detachably connected to a second syringe containing a second composition comprising forcing at least a portion of the first composition into the second syringe or forcing at least a portion of the second composition into the first syringe thereby providing a mixed composition.

10. The method of claim 9 wherein the first syringe comprises a male end portion comprising a locking ring configured to detachably connect a female end portion of a second syringe.

11. The method of claim 9 wherein the second syringe comprises a female end portion configured to detachably connect a locking ring of the first syringe.

12. The method of claim 11 wherein the female end portion of the second syringe is detachably connected to the male end portion of the first syringe via a locking ring such that the first composition and the second composition are in communication.

13. The method of claim 11, wherein the female end portion of the second syringe comprises exteriorly protruding members adapted to detachably engage a locking ring of the first syringe.

14. The method of claim 12 wherein the first composition and the second composition are each independently a fluid, a solid or a mixture thereof.

15. A method for administering a mixed composition to a patient wherein a first composition is contained within a first syringe, a second composition is contained within a second syringe, and the first syringe is detachably connected to the second syringe via a locking ring, comprising:
forcing at least a portion of the first composition into the second syringe or forcing at least a portion of the second composition into the first syringe, effective to provide a mixed composition;
disconnecting the first and second syringes;
connecting the locking ring of the first syringe to a discharge assembly configured to detachably connect to the locking ring;
inserting a portion of the discharge assembly into the patient; and
forcing at least a portion of the mixed composition through the discharge assembly effective to administer the portion of the mixed composition to the patient.

16. The method of claim 15 wherein the first syringe comprises a male end portion comprising a locking ring.

17. The method of claim 16 wherein the second syringe comprises a female end portion configured to detachably connect to the male end portion of the first syringe via the locking ring.

18. The method of claim 17 wherein the female end portion of the second syringe is connected to the male end portion of the first syringe via the locking ring such that the first composition and the second composition are in communication.

19. The method of claim 16, wherein the female end portion of the second syringe comprises exteriorly protruding members adapted to detachably engage the locking ring.

20. The method of claim 15 wherein the discharge assembly comprises a needle.

21. The method of claim 15 wherein the administration is by an intravenous, intramuscular, intraperitoneal, or subcutaneous route.

22. The method of claim 15 wherein the first composition and the second composition are each independently a fluid, a solid or a mixture thereof.
